(19) United States
(12) Patent Application Publication
(10) Pub. No.: US 2023/0162820 A1
(43) Pub. Date: May 25, 2023

(54) COMPUTING SYSTEM AND METHOD FOR RELEVANCY CLASSIFICATION OF CLINICAL DATA SETS USING KNOWLEDGE GRAPHS

(71) Applicant: Hyland Software, Inc., Westlake, OH (US)

(72) Inventors: Dan Matei, Westlake, OH (US); Iryna Gordon, Westlake, OH (US); Jennifer Tucker, Westlake, OH (US)

(21) Appl. No.: 17/988,986

(22) Filed: Nov. 17, 2022

Related U.S. Application Data
(60) Provisional application No. 63/282,762, filed on Nov. 24, 2021.

(51) Int. Cl. G16H 10/20; G16H 10/60; G16H 30/20; G06N 5/02

(52) U.S. Cl. CPC G16H 10/20; G16H 10/60; G16H 30/20; G06N 5/02

(57) ABSTRACT

A computing system receives a keyword, where the keyword corresponds to a tagged clinical data item in tagged clinical data items. The tagged clinical data items are generated based upon tags and rules included in the templates. The computing system identifies a seed node in a knowledge graph based upon the keyword, where nodes in the knowledge graph represent the templates and where edges in the knowledge graph represent relationships between the templates. The computing system identifies a subset of nodes in the knowledge graph that include the seed node or are connected to the seed node. The computing system identifies a subset of the tagged clinical data items based upon the subset of nodes and causes graphical data corresponding to the subset of the tagged clinical data items to be presented on a display.

### CROSS-REFERENCE TO RELATED APPLICATIONS

This application claims the benefit of priority to U.S. provisional application 63/282,762, filed on Nov. 24, 2021. That prior application is incorporated by reference herein in its entirety.

### BACKGROUND

Healthcare software (e.g., electronic health records (EHR) applications) have access to vast amounts of patient data for patients. For instance, patient data may be scattered across multiple disparate computing systems. Moreover, the patient data includes many different types of data, such as imaging studies, videos, clinical documentation, algorithmic results, and other types of ancillary clinical data. Conventional healthcare software struggles to identify information that is relevant to a healthcare worker. Conventional healthcare software also struggles to determine an optimal manner in which to display such information to the healthcare worker. These struggles are particularly acute in clinical study scenarios.

One approach utilized by conventional healthcare software is to utilize matching rules in order to identify and display patient data that is relevant to a healthcare worker. However, matching rules tend to be overly inclusive or overly exclusive. When a matching rule is overly inclusive, a conventional healthcare software application identifies and displays patient data that is not relevant to the healthcare worker. This is burdensome on the limited display resources available to the healthcare software. However, when a matching rule is overly exclusive, a conventional healthcare software may fail to identify patient data that is relevant to the healthcare worker. In such scenarios, the conventional healthcare software receives manual input from the user in order to search for the patient data that the healthcare worker requires, which is also burdensome on resources of the healthcare software.

### SUMMARY

The following is a brief summary of subject matter that is described in greater detail herein. This summary is not intended to be limiting as to the scope of the claims.

According to some embodiments, a computing system comprises a processor, a data store, and memory. The data stores templates, where each template in the templates comprises a tag and at least one rule. The data store also stores a knowledge graph. The knowledge graph comprises nodes and edges connecting the nodes, where the nodes represent the templates and the edges represent relationships between the templates. Each node comprises a respective tag corresponding to a respective template in the templates. The computing system obtains clinical data items of a patient. The computing system matches each clinical data item in the clinical data items to one or more templates in the templates based upon rules comprised by the templates. The computing system generates tagged clinical data items based upon the clinical data items and tags comprised by the templates upon the clinical data items being matched. Upon receiving a keyword, the computing system identifies a seed node in the knowledge graph based upon the keyword. The computing system identifies a subset of nodes in the knowledge graph based upon the seed node and a non-negative integer, where the subset of nodes includes the seed node and first nodes, and where each of the first nodes are connected to the seed node in the knowledge graph by no more than a number of edges equal to the non-negative integer. The computing system identifies a subset of the tagged clinical data items based upon first tags of the subset of nodes. The computing system causes graphical data corresponding to the subset of the tagged clinical data items to be presented on a display.

According to some embodiments, a computing system receives a selection of a first tagged clinical data item of a patient from amongst tagged clinical data items of the patient. The tagged clinical data items are generated based upon clinical data items from a plurality of electronic sources being matched to templates stored in a data store. Each template comprises a tag and at least one rule, where rules of the templates are matched to the clinical data items, thereby generating the tagged clinical data items. The computing system identifies a seed node in a knowledge graph stored in the data store based upon a first tag of the first tagged clinical data item. The knowledge graph comprises nodes and edges connecting the nodes, wherein the nodes represent the templates and the edges represent relationships between the templates. Each node comprises a respective tag corresponding to a respective template in the templates. The computing system identifies a subset of nodes in the knowledge graph based upon the seed node and a non-negative integer, where the subset of nodes includes the seed node and first nodes, and where each of the first nodes are connected to the seed node in the knowledge graph by no more than a number of edges equal to the non-negative integer. The computing system identifies a subset of the tagged clinical data items of the patient based upon first tags of the subset of nodes. The computing system causes graphical data corresponding to the subset of the tagged clinical data items to be presented on a display.

According to some embodiments, a computing system obtains clinical data items of a patient. The computing system matches each clinical data item in the clinical data items to one or more templates in templates stored in a data store based upon rules comprised by the templates. The computing system generates tagged clinical data items based upon the clinical data items and tags comprised by the templates upon the clinical data items being matched. The computing system receives a keyword from a computing device operated by a healthcare worker. The computing system identifies at least one seed node in a knowledge graph stored in the data store based upon the keyword. The knowledge graph comprises nodes and edges connecting the nodes. The nodes represent the templates and the edges represent relationships between the templates. Each node comprises a respective tag corresponding to a respective template in the templates. The computing system identifies a first subset of the tagged clinical data items based upon a tag of the at least one seed node, where each of the first subset of the tagged clinical data items includes the tag of the seed node. The computing system causes first graphical data corresponding to the first subset of the tagged clinical data items to be presented on a display of the computing device. Upon receiving an indication from the computing device, the computing system identifies a subset of nodes in the knowledge graph, where each node in the subset of nodes is connected to the at least one seed node via an edge. The computing system identifies a second subset of the tagged clinical data items based upon the subset of nodes. The computing system causes second graphical data corresponding to the second subset of the tagged clinical data items to be presented on the display concurrently with the first graphical data.

The above summary presents a simplified summary in order to provide a basic understanding of some aspects of the systems and/or methods discussed herein. This summary is not an extensive overview of the systems and/or methods discussed herein. It is not intended to identify key/critical items or to delineate the scope of such systems and/or methods. Its sole purpose is to present some concepts in a simplified form as a prelude to the more detailed description that is presented later.

### BRIEF DESCRIPTION OF THE DRAWINGS

FIG. 1 is a functional block diagram of an exemplary computing system that facilitates incremental search over clinical data items using a knowledge graph.

FIG. 2 is a detailed functional block diagram of the exemplary computing system illustrated in FIG. 1.

FIG. 3 illustrates exemplary knowledge graphs.

FIGS. 4A-E illustrate sequential views of an exemplary graphical user interface (GUI) that facilitates displaying relevant clinical data items to a healthcare worker.

FIG. 5 is a flow diagram that illustrates an exemplary methodology performed by a computing system for tagging clinical data items.

FIG. 6 is a flow diagram that illustrates an exemplary methodology performed by a computing system that facilitates displaying relevant clinical data items.

FIG. 7 illustrates an exemplary computing device.

### DETAILED DESCRIPTION

As noted above, conventional healthcare software utilizes rule-based approaches to identify and display relevant patient data for a patient to a healthcare worker. Such rule-based approaches tend to be overly inclusive or overly exclusive, which strains computing resources that are available to the healthcare software. Rule-based approaches tend to be particularly problematic in clinical study scenarios.

To address these issues, a computing system is described herein that is configured to classify (i.e., tag) clinical data items based upon templates, where the templates include a tag used for classification purposes and at least one rule. The computing system utilizes a keyword to locate seed nodes in a knowledge graph, where the knowledge graph comprises nodes representing the templates and edges representing relationships between the templates. The computing system identifies additional nodes in the knowledge graph that are connected to the seed nodes via one or more edges. Upon identifying the additional nodes, the computing system identifies corresponding clinical data items from the tagged clinical data items. The computing system presents graphical data on a display, where the graphical data corresponds to the tagged clinical data items. The computing system enables a healthcare worker to incrementally discover new information about a patient.

In an example operation, a computing system obtains clinical data items of a patient from a plurality of electronic sources. The clinical data items may include historical imaging studies, clinical reports, measurements, videos, etc. The clinical data items may include Digital Imaging and Communications in Medicine (DICOM) clinical data items and non-DICOM clinical data items. The computing system converts the non-DICOM clinical data items to DICOM format. The computing system matches the clinical data items to one or more templates, where a template comprises a tag (e.g., a name) and at least one rule. The templates may represent a body part, a medical facility, an imaging modality, a disease, etc. The templates may be user-defined. In one example, the tag is a body part such as “liver.” In another example, the tag is a disease such as “liver cancer.” The computing system evaluates a clinical data item against at least one rule in a template in order to match the clinical data item to the template. The computing system generates tagged clinical data items upon matching the clinical data items, where the tagged clinical data items include tags corresponding to templates to which the clinical data items are matched. In an example, the clinical data item is a liver image study and the template has a “liver” tag and a rule that indicates that the template is to be matched to clinical data items that include liver image study related metadata. In the example, the computing system tags the liver image study with the “liver” tag upon evaluating the liver image study against the rule in the template.

According to first embodiments (referred to herein as “prefetching”), subsequent to generating the tagged clinical data items, the computing system receives a keyword and a non-negative integer. The keyword may correspond to one or more tags of the tagged clinical data items. The computing system may receive the keyword and the non-negative integer as input from a healthcare worker. In an example, the keyword is “liver” and the non-negative integer is “two.” The computing system identifies a seed node (or more than one seed node) in a knowledge graph based upon the keyword. The knowledge graph comprises nodes and edges connecting the nodes, where the nodes represent the templates and where the edges represent relationships between the templates. Each node comprises a tag that corresponds to one of the templates. The knowledge graph may include user-defined nodes. In an example, the knowledge graph includes a first node representing a liver template that is connected to a second node representing an abdomen template due to the liver being part of the abdomen region of the human body, and a third node representing the pelvis that is connected to the second node due to the abdomen region of the human body being connected to the pelvis region of the human body. Upon identifying the seed node, the computing system identifies a subset of nodes in the knowledge graph, where the subset of nodes include the seed node and first nodes, where each of the first nodes are connected to the seed node by no more than a number of edges equal to the non-negative integer. Following the example given above in which the seed node in the knowledge graph corresponds to the liver, the computing system identifies the second node (as the first node representing the liver template is connected to the second node representing the abdomen template by one edge) and the third node (as the first node representing the liver template is connected to the third node representing the pelvis template by two edges). Based upon tags comprised by the subset of nodes, the computing system identifies a subset of the tagged clinical data items. In an example, the computing system identifies a liver image study, an abdomen clinical study, and a pelvis clinical study. The computing system causes graphical data corresponding to the subset of the tagged clinical data items to be presented on a display. In an example, the graphical data includes an identifier for the liver image study, an identifier for the abdomen clinical study, and an identifier for the pelvis clinical study. In another example, the graphical data includes an image from the liver image study, extracts from the abdomen clinical study, and extracts from the pelvis clinical study. According to embodiments, the graphical data is arranged in a timeline based upon associated dates of the subset of the tagged clinical data items.

According to second embodiments (referred to herein as “postfetching”), the computing system receives a keyword. The keyword may correspond to one or more tags of the tagged clinical data items. The computing system may receive the keyword as input from a healthcare worker. In an example, the keyword is “liver.” The computing system identifies a seed node (or more than one seed node) in the knowledge graph based upon the keyword. In an example, the knowledge graph includes a first node representing a liver template that is connected to a second node representing an abdomen template due to the liver being part of the abdomen region of the human body, and a third node representing the pelvis that is connected to the second node due to the abdomen region of the human body being connected to the pelvis region of the human body. Upon identifying the seed node, the computing system identifies a first subset of the tagged clinical data items that have corresponding tags matching the tag of the seed node. The computing system causes first graphical data corresponding to the first subset of the tagged clinical data items to be presented on a display. In an example, the first graphical data includes identifiers for liver image studies. The first graphical data may be in the form of a timeline. Subsequently, the computing system receives an indication that that healthcare worker has selected a “plus one” button. Upon receiving the indication, the computing system identifies a subset of nodes in the knowledge graph, where each node in the subset of nodes is connected to the seed node via an edge. In an example, the subset of nodes include a node corresponding to an abdomen template. The computing system identifies a second subset of the tagged clinical data items based upon tags comprised by the subset of nodes. In an example, the second subset of the tagged clinical data items include abdomen clinical studies. The computing system causes second graphical data corresponding to the second subset of the tagged clinical data items to be presented on the display concurrently with the first graphical data. In an example, the second graphical data includes identifiers for the abdomen clinical studies. The second graphical data may be included in the form of a timeline.

The above-described technologies present various advantages over conventional computer-implemented approaches to identifying and displaying relevant patient data to a healthcare worker. First, by leveraging the knowledge graph described above, the computing system is able to accurately identify relevant clinical data items for display without being limited by overly inclusive or overly exclusive matching rules. Second, as the knowledge graph is modifiable by a user, the above-described technologies enable the automatic surfacing of clinical data items, even when the clinical data items correspond to different types of data. For instance, the technologies described above can simultaneously discover and display images from an image study along with clinical reports. Third, via the incremental approach of displaying clinical data items described above, the technologies described herein efficiently utilize limited display space. For instance, the incremental approach described above ensures that the computing system displays relevant clinical data items to the healthcare worker while not overcrowding available display space with irrelevant clinical data items.

Various technologies pertaining to classification of clinical data items based upon knowledge graphs are now described with reference to the drawings, wherein like reference numerals are used to refer to like items throughout. In the following description, for purposes of explanation, numerous specific details are set forth in order to provide a thorough understanding of one or more aspects. It may be evident, however, that such aspect(s) may be practiced without these specific details. In other instances, well-known structures and devices are shown in block diagram form in order to facilitate describing one or more aspects. Further, it is to be understood that functionality that is described as being carried out by certain system components may be performed by multiple components. Similarly, for instance, a component may be configured to perform functionality that is described as being carried out by multiple components.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

Further, as used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary” is intended to mean serving as an illustration or example of something, and is not intended to indicate a preference.

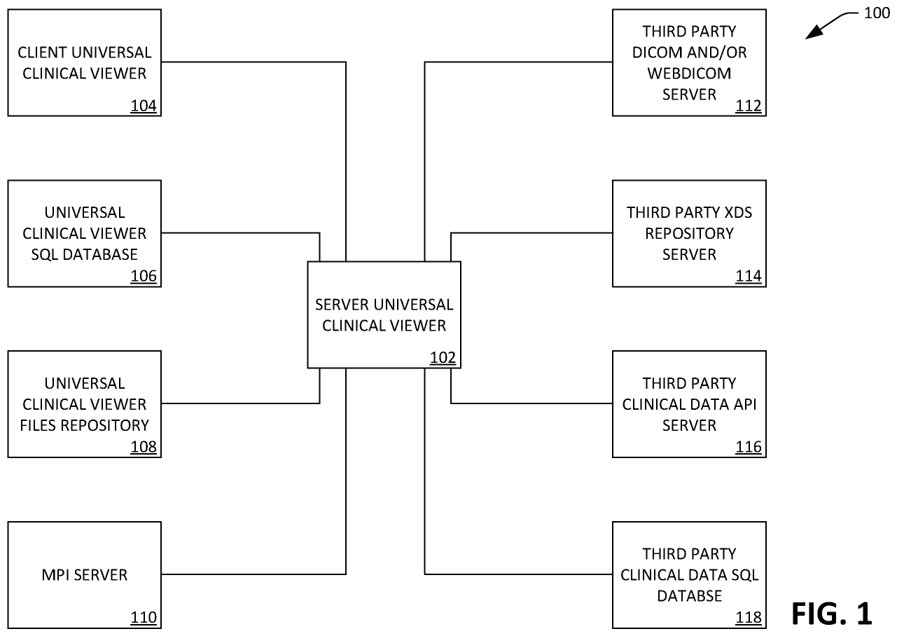

With reference to FIG. 1, an exemplary computing system 100 that facilitates incremental search over clinical data items using a knowledge graph is illustrated. The computing system 100 includes a server universal clinical viewer 102. As will be explained in greater detail below, the server universal clinical viewer 102 is configured to tag clinical data items based upon tags comprised by templates, where the server universal clinical viewer 102 tags the clinical data items by executing one or more rules comprised by the templates. The server universal clinical viewer 102 is further configured to identify nodes in a knowledge graph based upon a keyword, where nodes of the knowledge graph represent the templates and edges in the knowledge graph represent relationships between the templates. The server universal clinical viewer 102 is also configured to identify tagged clinical data items based upon the nodes identified in the knowledge graph. The server universal clinical viewer 102 may be or include a server electronic health records (EHR) application.

The computing system 100 further includes a client universal clinical viewer 104. The client universal clinical viewer 104 is configured to display graphical data to a user, where the graphical data corresponds to the tagged clinical data items identified by the server universal clinical viewer 102 using the knowledge graph. As will be explained in greater detail below, the client universal clinical viewer 104 is also configured to enable the user to incrementally select tagged clinical data items for display. The client universal clinical viewer 104 may be or include a client EHR application.

The computing system 100 includes a universal clinical viewer Structured Query Language (SQL) database 106. The universal clinical viewer SQL database 106 stores tagged clinical data items that are tagged by the server universal clinical viewer 102. The server universal clinical viewer 102 generates the tagged clinical data items based upon clinical data items received from a plurality of electronic sources. In an example, the universal clinical viewer SQL database 106 includes a table, and the table comprises a row corresponding to the (tagged) clinical data item and at least two columns. A first column in the at least two column stores a value for the (tagged) clinical data item. A second column in the at least two columns stores a tag. According to embodiments in which metadata for a clinical data item (and not the clinical data item itself) is stored in the universal clinical viewer SQL database 106, the at least two columns include a third column, where the third column includes an identifier for a storage location of the clinical data item. According to embodiments, another relational database may be utilized in place of the universal clinical viewer SQL database 106. The server universal clinical viewer 102 may be in communication with the universal clinical viewer SQL database 106 by way of a network.

The computing system 100 includes a universal clinical viewer files repository 108. According to some embodiments, the universal clinical viewer files repository 108 stores tagged clinical data items. According to other items, the universal clinical viewer files repository stores (untagged) clinical data items, where the tags of the clinical data items are located in the universal clinical viewer SQL database 106. The universal clinical viewer files repository 108 may be a non-relational database. The server universal clinical viewer 102 may be in communication with the universal clinical viewer files repository 108 by way of a network.

The computing system 100 may include a master patient index (MPI) server 110. The MPI server 110 includes a plurality of medical record numbers (MRNs) of a patient, where the plurality of MRNs are used by different healthcare enterprises, different healthcare software systems (e.g., different EHRs), and/or different patient identity domains to identify the patient. The server universal clinical viewer 102 may query the MPI server 110 with a MRN of a patient in order to obtain the plurality of MRNs. The server universal clinical viewer 102 may utilize the plurality of MRNs to obtain clinical data items of the patient from the different healthcare enterprises, the different healthcare software systems, and/or the different patient identity domains.

The computing system 100 includes a third party Digital Imaging and Communications in Medicine (DICOM) or webDICOM server 112. DICOM is a standard for communication and management of medical imaging information and related data. WebDICOM is a standard for web-based medical imaging. The server universal clinical viewer 102 connects to the third party DICOM and/or webDICOM server 112 using standard DICOM and/or webDICOM protocols. The third party DICOM and/or webDICOM server 112 may be or include Picture and Archiving communication systems (PACS), vendor neutral archives (VNA), or acquisition modalities such as computed tomography (CT) scanners, ultrasound (US) machines, etc. The third party DICOM and/or webDICOM server 112 provides a patient history as study level metadata.

The computing system 100 includes a third party Cross-Enterprise Document Sharing (XDS) repository server 114. XDS is a system of standards for cataloging and sharing patient records across healthcare enterprises. The server universal clinical viewer 102 connects the third party XDS repository server 114 using standard XDS specification communication protocols. The third party XDS repository server 114 provides document information corresponding to a patient history.

The computing system 100 may include a third party clinical data application programing interface (API) server 116. The third party clinical data API server 116 may provide the server universal clinical viewer 102 with clinical data items that are in a proprietary format. The server universal clinical viewer 102 connects with the third party clinical data API server 116 using proprietary data source adapters that are implemented following third party system specifications.

The computing system 100 may include a third party clinical data SQL database 118. The third party clinical data SQL database 118 stores clinical data items in tables. The server universal clinical viewer 102 connects with the third party clinical data SQL database 118 using proprietary data source adapters that are implemented following third party system specifications. Alternatively, the server universal clinical viewer 102 connects with the third party clinical data SQL database 118 using the third party clinical data API server 116 as an intermediary.

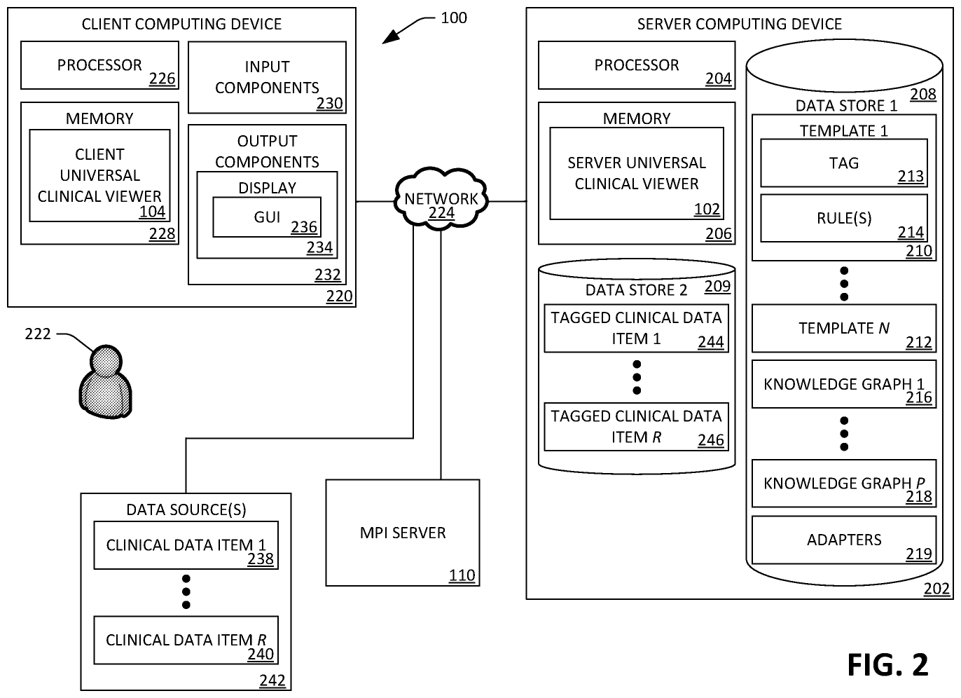

Referring now to FIG. 2, a detailed functional block diagram of the computing system 100 is illustrated. The computing system 100 includes a server computing device 202. According to embodiments, the server computing device 202 is a cloud-based computing platform. According to embodiments, the server computing device 202 is under control of a healthcare enterprise.

The server computing device 202 includes a processor 204 and memory 206, where the memory 206 has the server universal clinical viewer 102 loaded therein (described above). The processor 204 executes the server universal clinical viewer 102 to perform functionality that will be explained in detail below.

The server computing device 202 includes a first data store 208. The server computing device may also include a second data store 209. The first data store 208 stores a first template 210 and an Nth template 212, where Nis a positive integer greater than one. The first template 210 and the Nth template 212 are collectively referred to herein as “the templates 210-212” or “the plurality of templates 210-212.” The templates 210-212 may include pre-defined templates and/or user-defined templates. Each template in the templates 210-212 may represent a body part, a body region, a medical facility, a medical department within a medical facility, an imaging modality, a piece of medical equipment, a clinical observation, or a disease (where the disease affects a body part or a body region).

The first template 210 comprises a tag 213. In general, the tag 213 is a classification of medical related data. The tag 213 may be a name of a body part (or a name of a body region or system), a name of a medical facility, a name of a department within the medical facility, a name of an imaging modality, a name of a piece of medical equipment, or a name of a disease (or a name of a clinical observation). In an example, a body part tag is “CHEST.” In another example, a department tag is “EMERGENCY_ROOM.” In yet another example, a modality tag is “XRAY”. In yet another example, an equipment specific tag is “US_MODALITY_MODEL2.” In a further example, a disease tag is “CORONARY_ARTERY_CALCIFICATION.”

The first template 210 comprises a rule 214 (or more than one rule). The rule 214 includes an evaluation criteria. The rule 214 may include a comparison value. The evaluation criteria may include “contains,” “contains any of,” “exact match,” “bigger,” “smaller,” “exists,” “does not exist,” “is null,” or “is empty.” The rule 214 may be a regular expression. The comparison value may be a number or a string. According to embodiments, the comparison value is a value field of a DICOM attribute. According to embodiments, the server universal clinical viewer 102 evaluates a DICOM tag of a DICOM clinical data item against a DICOM attribute (i.e., a comparison value) specified by the rule 214, including private and hierarchical sequences included in the DICOM tag. It is to be understood that the rule 214 may be grouped together in the first template 210 with other rules in the first template 210 using logical operators such as “AND, “OR,” or “XOR.” The Nth template 212 includes components similar to that of the first template 210 (e.g., a tag and one or more rules, where the tag and/or the one or more rules differ from that of the first template 210).

The data store 208 further stores a first knowledge graph 216 and a Pth knowledge graph 218, where P is a positive integer greater than one. The first knowledge graph 216 and the Pth knowledge graph 218 are collectively referred to herein as “the knowledge graphs 216-218” or “the plurality of knowledge graphs 216-218.” The knowledge graphs 216-218 may be user-editable such that users may add user-defined nodes (representing user-defined templates) to the knowledge graphs 216-218.

The first knowledge graph 216 comprises nodes and edges connecting the nodes, where each node represents a template (e.g., the first template 210, the Nth template 212, etc.) and where the edges represent relationships between the templates 210-212 (e.g., a relationship between the first template 210 and the Nth template 212). Each node in the first knowledge graph 216 comprises a tag corresponding to one of the templates 210-212. In an example, the first knowledge graph 216 includes a node representing the first template 210, and the node includes the tag 213. According to embodiments, the edges in the knowledge graph 216 are directed edges. In an example, each node in the first knowledge graph 216 represents a body part template.

The Pth knowledge graph 218 is similar to the first knowledge graph 216. For instance, the Pth knowledge graph 218 comprises nodes and edges connecting the nodes; however, nodes in the Pth knowledge graph 218 represent different templates than those of the first knowledge graph 216. In an example, each node in the Pth knowledge graph 218 represents a disease template.

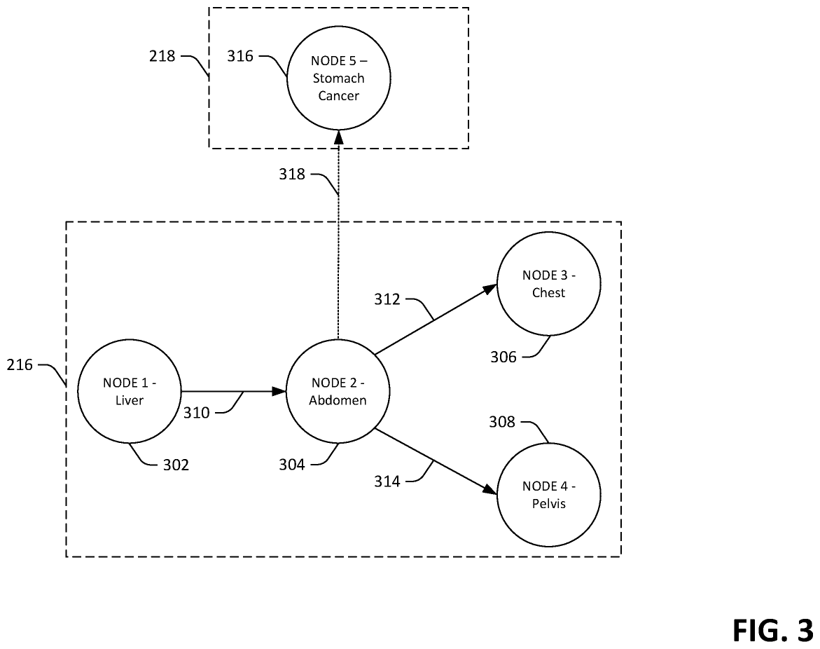

Referring now to FIG. 3, an example of the first knowledge graph 216 and the Pth knowledge graph 218 is depicted. In the example depicted in FIG. 3, the first knowledge graph 216 includes a first node 302 that represents a template assigned to a liver body part, a second node 304 that represents a template assigned to an abdomen body region, a third node 306 that represents a template assigned to a chest body region, and a fourth node 308 that represents a template assigned to a pelvis body region. The first node 302 is connected to the second node 304 by a first edge 310 due to the liver being a part of the abdomen body region. Similarly, the second node 304 is connected to the third node 306 by a second edge 312 due to the abdomen body region being located adjacent to the chest body region. Likewise, the second node 304 is connected to the fourth node 308 by a third edge 314 due to the abdomen body region being located adjacent to the pelvis body region. It is to be understood that the first knowledge graph 216 may include may many other nodes and edges.

In the example depicted in FIG. 3, the Pth knowledge graph 218 includes a fifth node 316 that represents a template assigned to stomach cancer. The Pth knowledge graph 218 may also include many other nodes and edges (not depicted in FIG. 3). In an example, the many other nodes in the Pth knowledge graph 218 represent templates assigned to different diseases. As depicted in FIG. 3, the second node 304 is connected to the fifth node 316 by an edge 318. The server universal clinical viewer 102 may connect the first knowledge graph 216 and the Pth knowledge graph 218 via the edge 318 based upon input set forth by a user. For instance, while the user may initially be interested in clinical data items relating to the liver, the user may also be interested in clinical data items relating to stomach cancer. According to embodiments, the first knowledge graph 216 and the Pth knowledge graph 218 may be considered a single knowledge graph due to the edge 318 connecting the first knowledge graph 216 and the Pth knowledge graph 218. In this manner, when the server universal clinical viewer 102 walks the first knowledge graph 216, the server universal clinical viewer 102 may discover additional nodes representing information of interest to the user.

Referring back to FIG. 2, the data store 208 further includes adapters 219. The server universal clinical viewer 102 utilizes the adapters 219 to convert non-DICOM clinical data items to DICOM attributes (explained in greater detail below).

The computing system 100 includes a client computing device 220 that is operated by a healthcare worker 222. According to embodiments, the client computing device 220 is a desktop computing device, a laptop computing device, a tablet computing device, or a smartphone. The client computing device 220 is in communication with the server computing device 202 by way of a network 224 (e.g., the Internet, intranet, etc.).

The client computing device 220 comprises a processor 226 and memory 228. The memory 228 has the client universal clinical viewer 104 loaded therein. The processor 226 executes the client universal clinical viewer 104 to perform functionality that will be explained in detail below.

The client computing device 220 includes input components 230 that enable the healthcare worker 222 to set forth input to the client computing device 220. The input components 230 include one or more of a mouse, a keyboard, a touchscreen, a trackpad, a scroll wheel, a camera, a video camera, or a microphone. The client computing device 220 includes output components 232 that output information to the healthcare worker 222. The output components 232 include a display 234. The display 234 may be a touchscreen display. The client universal clinical viewer 104 presents a graphical user interface (GUI) 236 on the display 234, where the GUI 236 includes clinical data items (to be explained in greater detail below). Although not depicted in FIG. 2, the output components 232 may also include a speaker that outputs audible noises.

With reference generally now to FIGS. 1 and 2, operation of the computing system 100 is now set forth. The server universal clinical viewer 102 obtains, over the network 224, a first clinical data item 238 and an Rth clinical data item 240 from one or more data sources 242, where R is a positive integer greater than one. The first clinical data item 238 and the Rth clinical data item 240 are collectively referred to herein as “the clinical data items 238-240” or “the plurality of clinical data items 238-240.” The clinical data items 238-240 relate to a patient. According to embodiments, the server universal clinical viewer 102 performs a federated search over the one or more data sources 242 based upon an identifier for the patient.

In one example, the client universal clinical viewer 104 receives an identifier for the patient (e.g., a MRN) as input by the healthcare worker 222. The client universal clinical viewer 104 transmits the identifier for the patient to the server universal clinical viewer 102. Upon receiving the identifier for the patient, the server universal clinical viewer 102 obtains the clinical data items 238-240 of the patient from the one or more data sources 242. In another example, the server clinical viewer 102 receives a list of identifiers for patients, where the list includes the identifier for the patient. The server clinical viewer 102 obtains the clinical data items 238-240 of the patient upon receiving the list.

According to embodiments, the server universal clinical viewer 102 stores the clinical data items 238-240 in the universal clinical viewer files repository 108. According to the embodiments, the server universal clinical viewer 102 causes an entry (e.g., a row) to be created in the universal clinical viewer SQL database 106 for each of the clinical data items 238-240. An entry for the first clinical data item 238 may include (in different columns of a table) a storage location of the first clinical data item 238 in the universal clinical viewer files repository 108, metadata for the first clinical data item 238, and/or some or all of the first clinical data item 238 itself.

According to embodiments, upon receiving a MRN of the patient, the server universal clinical viewer 102 transmits the MRN of the patient to the MPI server 110 by way of the network 224. The MPI server identifies a plurality of MRNs of the patient using the MRN, where the plurality of MRNs are used by different healthcare enterprises, different healthcare software, and/or different healthcare domains to identify the patient. The MPI server 110 transmits the plurality of MRNs to the server universal clinical viewer 102 by way of the network 224. The server universal clinical viewer 102 utilizes the plurality of MRNs to obtain the clinical data items 238-240 from the one or more data sources 242.

The one or more data sources 242 may be or include the third party DICOM and/or webDICOM server 112, the third party XDS repository server 114, the third party clinical data API server 116, and/or the third party clinical data SQL database 118. The one or more data sources 242 may include one or more EHRs or one or more Health Information Exchanges (HIEs).

The clinical data items 238-240 include one or more of historical imaging studies, clinical reports, admission forms, radiation reports, results obtained as output of an algorithm or an artificial intelligence (AI) model, measurements taken by healthcare workers or instruments employment by healthcare workers, videos, or clinical portable document format (PDF) documents. The clinical data items 238-240 include DICOM clinical data items and non-DICOM clinical data items. A DICOM clinical data item comprises DICOM attributes.

For certain types of items in the clinical data items 238-240, the server universal clinical viewer 102 performs additional queries for a configurable number of historical data sets, depending on modality, service-object-pair (SOP) class, or other criteria. The server universal clinical viewer 102 may also utilize configurable limits in performing searches, such as limiting the searches to items in the clinical data items 238-240 that were created within a particular time range. The server universal clinical viewer 102 may also limit access to the one or more data sources 242 to groups of particular users. The server universal clinical viewer 102 may also gather metadata for other types of searches, such as a user performing a search in a patient search window, following evaluation of worklists, or for prefetch/postfetch purposes.

Upon obtaining the clinical data items 238-240, the server universal clinical viewer 102 converts non-DICOM clinical data items in the clinical data items 238-240 to DICOM clinical data items using the adapters 219. The server universal clinical viewer 102 may also normalize the (converted) DICOM clinical data items. After conversion, the clinical data items 238-240 are a collection of DICOM attributes.

The server universal clinical viewer 102 matches each of the clinical data items 238-240 to one or more of the templates 210-212 based upon rules comprised by the one or more of the templates 210-212. In an example, the server universal clinical viewer 102 evaluates the first clinical data item 238 against a comparison value using evaluation criteria in order to match the first clinical data item 238 to the first template 210, where the comparison value and the evaluation criteria are included in the rule 214 of the first template 210. In another example, the server universal clinical viewer 102 evaluates the first clinical data item 238 using the evaluation criteria in order to match the first clinical data item 238 to the first template 210. In yet another example, the server universal clinical viewer 102 compares a first DICOM attribute in the first clinical data item 238 to a second DICOM attribute based upon evaluation criteria, where the second DICOM attribute and the evaluation criteria are included in the rule 214 of the first template 210. For instance, the evaluation criteria may be “contains” and the second DICOM attribute may be “chest.” When the first DICOM attribute in the first clinical data item 238 corresponds to “chest,” the server universal clinical viewer matches the first clinical data item 238 to the first template 210. It is to be understood that the server universal clinical viewer 102 may match the first clinical data item 238 to more than one template in the templates 210-212.

The server universal clinical viewer 102 generates a first tagged clinical data item 244 upon matching the first clinical data item 238 to one or more of the templates 210-212, where the first tagged clinical data item 244 includes one or more tags corresponding to the one or more templates to which the first clinical data item 238 has been matched. Following the example given above, the first tagged clinical data item 246 includes the tag “chest” and DICOM attributes. The server universal clinical viewer 102 also generates a Rth tagged clinical data item 246 upon matching the Rth clinical data item 240 to one or more of the templates 210-212 in a manner similar to that described above. The first tagged clinical data item 244 and the Rth tagged clinical data item 246 are collectively referred to herein as “the tagged clinical data items 244-246” or “the plurality of tagged clinical data items 244-246.”

According to embodiments, the server universal clinical viewer 102 stores the tagged clinical data items 244-246 in the data store 209. According to the embodiments, each of the tagged clinical data items 244 include one or more tags and one or more DICOM attributes. According to the embodiments, when a tagged clinical data item was originally in non-DICOM format and was subsequently converted to DICOM format, the tagged clinical data item stored in the data store 209 includes a link to the original (non-DICOM) clinical data item. In an example, the original (non-DICOM) clinical data item is stored in the universal clinical viewer files repository 108.

According to other embodiments, the universal clinical viewer SQL database 106 includes an entry (e.g., a row) for each of the tagged clinical data items 244-246, where an entry for a tagged clinical data item includes one or more tags, DICOM attributes, and a link to a storage location of a clinical data item in the universal clinical viewer files repository 108, where the tagged clinical data item was generated from the clinical data item. The server universal clinical viewer 102 accesses the entry in the universal clinical viewer SQL database 106 in order to locate the clinical data item in the universal clinical viewer files repository 108 (e.g., after discovering the clinical data item via searching one of the knowledge graphs 216-218, explained below). The server universal clinical viewer 102 may transmit the clinical data item to the client universal clinical 104 for presentment on the display 234.

The tagged clinical data items 244-246 may be used for several purposes. In one example, the server universal clinical viewer 102 transmits identifiers for the tagged clinical data items 244-246 to the client universal clinical viewer 104, whereupon the client universal clinical viewer 104 presents the identifiers for the tagged clinical data items 244-246 as a column in a patient search results window, where the identifiers for the tagged clinical data items 244-246 are utilized as criteria for prefetching. The identifiers for the tagged clinical data items 244-246 may be utilized as keywords for surfacing relevant patient data. In another example, the tagged clinical data items 244-246 are used for hanging protocols comparison views. In a further example, the tagged clinical data items 244-246 are used for patient timeline information filtering. In yet another example, the tagged clinical data items 244-246 are used for relevancy evaluation using one or more of the knowledge graphs 216-218.

According to some embodiments, the server universal clinical viewer 102 generates the tagged clinical data items 244-246 “online,” that is, the server universal clinical viewer 102 generates the tagged clinical data items 244-246 in real-time responsive to receiving an identifier for a patient from the client universal clinical viewer 104 that is set forth by the healthcare worker 222. According to other embodiments, the server universal clinical viewer 102 generates the tagged clinical data items 244-246 “offline,” that is, the server universal clinical viewer 102 generates the tagged clinical data items 244-246 at a predefined interval (e.g., nightly).

The server universal clinical viewer 102 receives a keyword, where the keyword may correspond to a tag of one of the templates 210-212. The keyword may also correspond to one of the tagged clinical data items 244-246. In an example, the client universal clinical viewer 104 receives the keyword as input from the healthcare worker 222. The client universal clinical viewer 104 transmits the keyword to the server universal clinical viewer 102. According to other embodiments, the client universal clinical viewer 104 displays a list of keywords within the GUI 236. The client universal clinical viewer 104 receives a selection of the keyword as input from the healthcare worker 222.

Upon receiving the keyword, the server universal clinical viewer 102 identifies a seed node (or more than one seed node) in one or more of the knowledge graphs 216-218 based upon the keyword. In an example, the keyword is “liver.” In an example, the server universal clinical viewer 102 identifies the seed node based upon a tag of the seed node matching the keyword.

According to first embodiments (referred to herein as “prefetching”), the server universal clinical viewer 102 identifies a subset of nodes in the knowledge graph 216 (and/or the Pth knowledge graph 218) based upon the seed node and a non-negative integer (e.g., zero, one, two, three, etc.). In one example, the non-negative integer is received along with the keyword from the client universal clinical viewer 104. In another example, the non-negative integer is pre-defined. The subset of nodes include the seed node and first nodes. Each of the first nodes are connected to the seed node in the first knowledge graph 216 (and/or the Pth knowledge graph 218) by no more than a number of edges equal to the non-negative integer. When the non-negative integer is zero, the subset of nodes includes only the seed node. In an example with reference to FIG. 3, when the seed node is the first node 302 (corresponding to a liver template) and the non-negative integer is one, the subset of nodes includes the first node 302 and the second node 304, as the first node 302 is connected to the second node 304 by one edge (the first edge 310). In another example with reference to FIG. 3, when the seed node is the first node 302 and the non-negative integer is two, the subset of nodes include the first node 302, the second node 304 (due to a connection by the first edge 310), as well as the third node 306, the fourth node 308, and the fifth node 316. For instance, the third node 306 is included in the subset of nodes as the first node 302 (the seed node) is connected to the third node 306 by two edges: the first edge 310 and the second edge 312. The server universal clinical viewer 102 identifies a subset of the tagged clinical data items 244-246 based upon tags of the subset of nodes. In an example, the server universal clinical viewer 102 executes a search over the tagged clinical data items 244-246 based upon the tags of the subset of nodes. The search produces search results that include the subset of the tagged clinical data items 244-246.

The first embodiments described above are useful in prefetching and hanging protocols scenarios, as the server universal clinical viewer 102 can automatically identify relevant clinical data items by deciding the relevancy distance (reflected in the non-negative integer described above) to form a seed study. For instance, if a “liver” study is loaded and hanging protocols define a relevancy distance of “two”, the server universal clinical viewer selects clinical data items corresponding to the “liver” (distance zero), “abdomen” (distance one), “chest” (distance two), and “pelvis” (distance two).

The server universal clinical viewer 102 transmits graphical data corresponding to the subset of the tagged clinical data items 244-246 to the client universal clinical viewer 104, whereupon the client universal clinical viewer 104 presents the graphical data within the GUI 236. In one example, the graphical data includes identifiers for each of the subset of the tagged clinical data items 244-246. It is to be understood that the identifiers for each of the subset of the tagged clinical data items may be of different types. For instance, the graphical data may include an identifier for an image and an identifier for a clinical report. In another example, the graphical data includes images corresponding to each of the subset of the tagged clinical data items 244-246.

In an example, the subset of the tagged clinical data items 244-246 includes the first tagged clinical data item 244 and the first tagged clinical data item 244 represents an image, where the image is stored in the universal clinical viewer files repository 108. The server universal clinical viewer 102 utilizes an identifier a storage location of the image (included in the first tagged clinical data item 244) in order to locate the image in the universal clinical viewer files repository 108. The server universal clinical viewer 102 then includes the image in the graphical data. The server universal clinical viewer 102 may access other types of data (e.g., clinical reports) in a similar manner.

According to second embodiments (referred to herein as “postfetching”), the server universal clinical viewer 102 initially identifies the seed node (or seed nodes) in the first knowledge graph 216 (and/or the Pth knowledge graph 218) based upon the keyword. The server universal clinical viewer 102 identifies a first subset of the tagged clinical data items 244-246 based upon a tag of the seed node (or seed nodes), where each of the first subset of the tagged clinical data items 244-246 include the tag of the seed node. In an example, the server universal clinical viewer 102 executes a first search over the tagged clinical data items 244-246 based upon the tag of the seed node. The first search produces first search results that include the first subset of the tagged clinical data items 244-246. The server universal clinical viewer 102 transmits first graphical data corresponding to the first subset of the tagged clinical data items 244-246 to the client universal clinical viewer 104, whereupon the client universal clinical viewer 104 presents the first graphical data within the GUI 236. In an example in which the keyword is liver, the first graphical data includes identifiers for images of a liver of the patient. Upon receiving a selection of a button shown on the GUI 236, the client universal clinical viewer 104 transmits an indication to the server universal clinical viewer 102. Upon receiving the indication, the server universal clinical viewer 102 identifies a subset of nodes in the first knowledge graph 216 (and/or the Pth knowledge graph 218), where each node in the subset of nodes is connected to the seed node(s) via an edge. The server universal clinical viewer 102 identifies a second subset of the tagged clinical data items 244-246 based upon tags of the subset of nodes. In an example, the server universal clinical viewer 102 executes a second search over the tagged clinical data items 244-246 based upon tags of the subset of nodes. The second search produces second search results that include the second subset of the tagged clinical data items 244-246. The server universal clinical viewer 102 transmits second graphical data to the client universal clinical viewer 104, whereupon the client universal clinical viewer 104 presents the second graphical data within the GUI 236 concurrently with the first graphical data. Following the example given above, the second graphical data includes identifiers for abdomen studies. In this manner, the computing system 100 is able to incrementally expand the amount clinical data items displayed to the healthcare worker 222 based upon input from the healthcare worker, thus preserving display space while ensuring that the healthcare worker is presented with relevant information.

According to embodiments, each of the subset of the tagged clinical data items 244-246 includes an associated date (e.g., a date on which an image was taken, a date on which a clinical report was generated, etc.). According to the embodiments, the graphical data (or the first graphical data or the second graphical data) comprises a timeline that includes the identifiers for each of the subset of the tagged clinical data items, where such identifiers are chronologically (or reverse chronologically) arranged based upon the associated dates.

According to embodiments, the server universal clinical viewer 102 receives a user-defined template from the client universal clinical viewer 104, where the user-defined template includes a user-defined tag and at least one user-defined rule. The server universal clinical viewer 102 also receives a selection of a node in the first knowledge graph 216 (or the Pth knowledge graph 218). The server universal clinical viewer 102 generates a user-defined node in the first knowledge graph 216, where the user-defined node is connected to the node in the first knowledge graph 216.

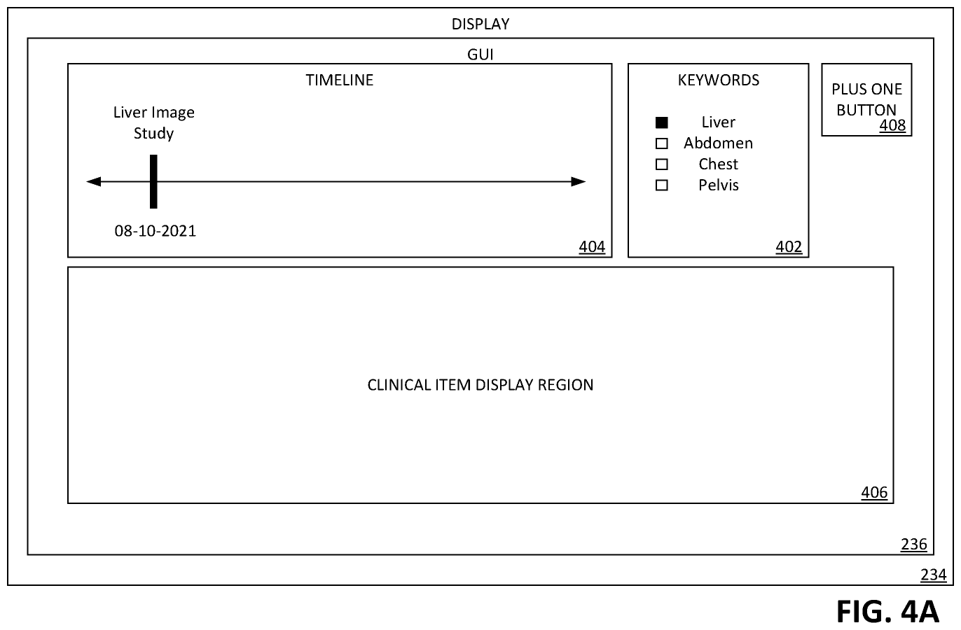

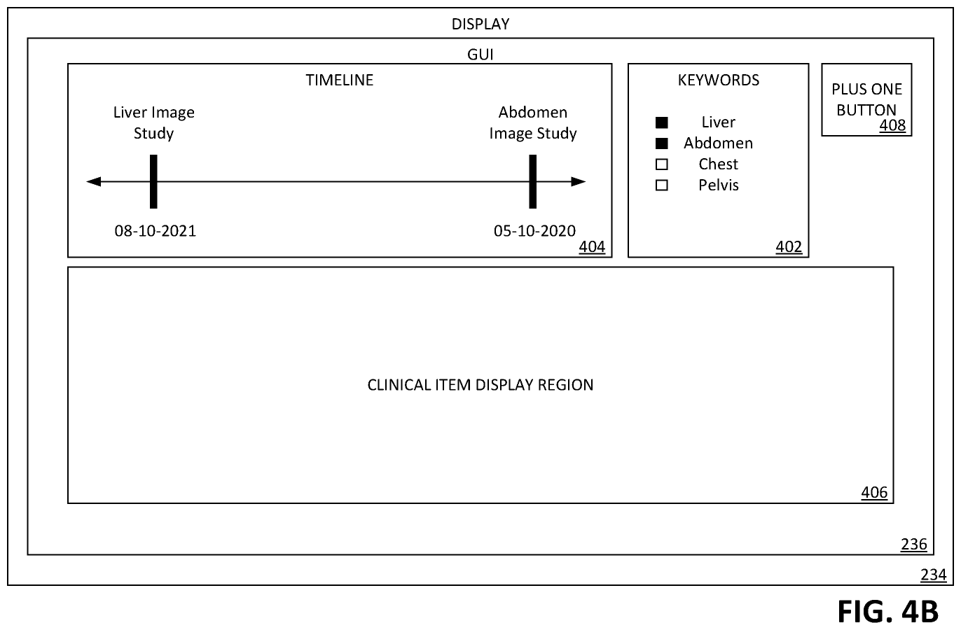

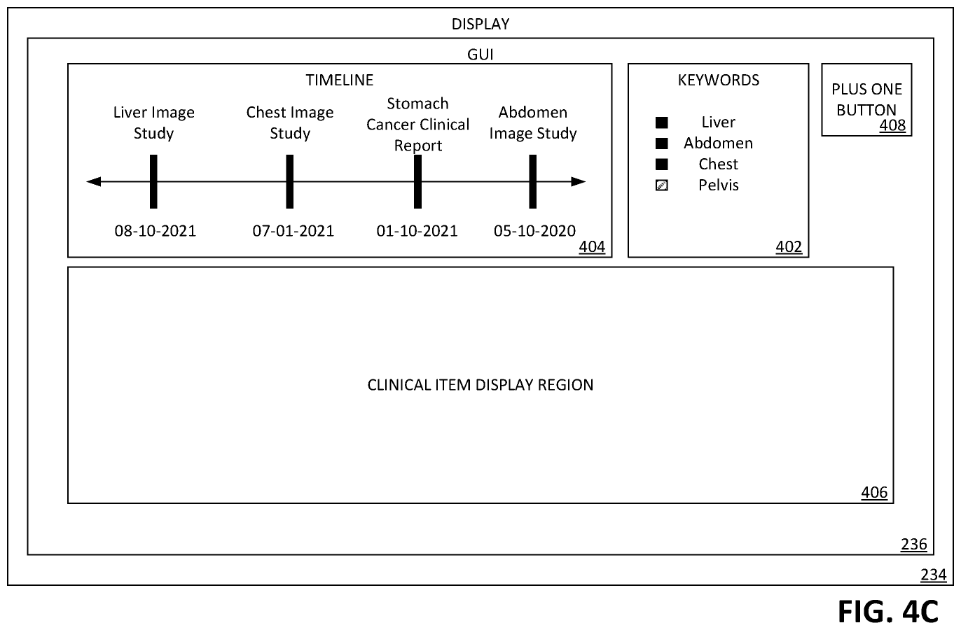

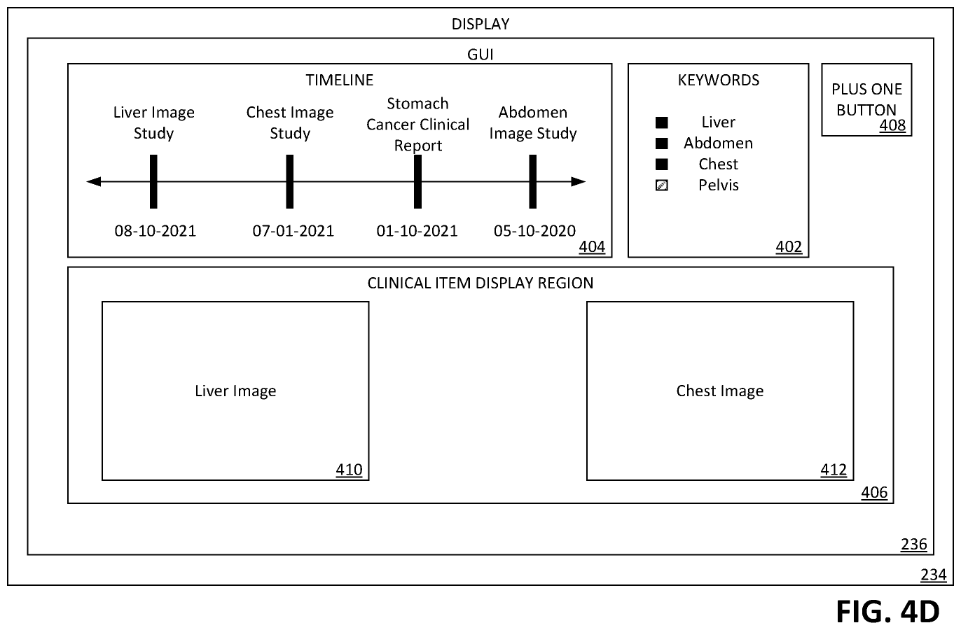

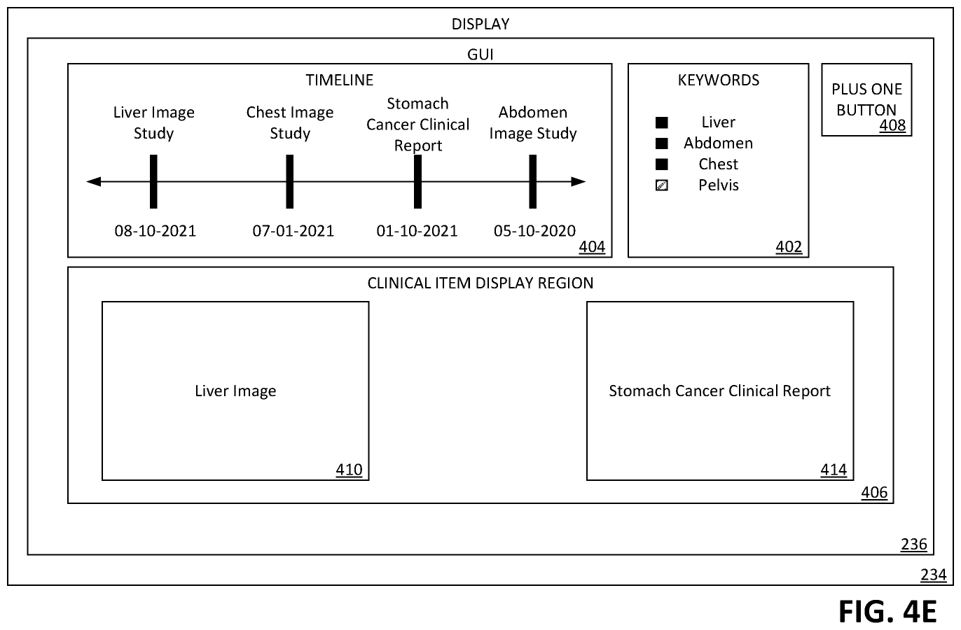

FIGS. 4A-D illustrate example states of the GUI 236 that is presented by the client universal clinical viewer 104. With reference now to FIG. 4A, the GUI 236 is depicted after the healthcare worker 222 has selected the “liver” keyword. In the example depicted in FIG. 4A, the seed node described above includes a “liver” tag. The GUI 236 includes a keyword region 402 that includes keywords, where the keywords correspond to tags of the templates 210-212. The GUI 236 further includes a timeline 404, where the timeline 404 includes an identifier for a liver image study (a clinical data item) and a date of the liver image study. The server universal clinical viewer 102 has identified the liver image study using the processes described above. The GUI 236 also includes a clinical item display region 406. In an example, when the identifier for the liver image study is selected, the client universal clinical viewer 104 causes one or more images from the liver image study to be presented in the clinical item display region 406. Alternatively, the client universal clinical viewer 104 may automatically display the one or more images upon the liver image study being identified by the server universal clinical viewer 102 using the processes described above. The GUI 236 further includes a plus one button 408. When the plus one button 408 is selected by the healthcare worker 222, the client universal clinical viewer 104 transmits an indication to the server universal clinical viewer 102, which causes the server universal clinical viewer 102 to perform the processes described above with respect to the second embodiments to identify additional nodes in the knowledge graph 216 (and/or the Pth knowledge graph 218), identify corresponding tagged clinical data items based upon the additional nodes, and transmit graphical data to the client universal clinical viewer 102, where the graphical data is based upon corresponding tagged clinical data items.

With reference now to FIG. 4B, the GUI 236 is depicted after the plus one button 408 is selected by the healthcare worker 222. As described above, the server universal clinical viewer 102 identifies abdomen clinical data items based upon the first knowledge graph 216 upon the plus one button 408 being selected. As such, “abdomen” is now selected in the keyword region 402. The timeline 404 now includes an identifier for an abdomen image study.

Referring now to FIG. 4C, the GUI 236 is depicted after the plus one button 408 is selected once again by the healthcare worker 222. As described above, the server universal clinical viewer 102 identifies chest clinical data items based upon the first knowledge graph 216 upon the plus one button 408 being selected. As such, “chest” is now selected in the keyword region 402. The server universal clinical viewer 102 has also searched the tagged clinical data items 244-246 using the pelvis tag, but has not located any such items (indicated by cross hatching in FIG. 4C). Additionally, based upon the edge 318 connecting the second node 304 (abdomen template) in the first knowledge graph 216 to the fifth node (stomach cancer template) in the Pth knowledge graph 218, the server universal clinical viewer 102 has identified a stomach cancer clinical data item in the tagged clinical data items 244-246. As such, the timeline 404 now includes an identifier for a chest image study of the patient and an identifier for a stomach cancer clinical report of the patient.

Turning now to FIG. 4D, the GUI 236 is depicted after the client universal clinical viewer 104 receives selections of the identifier for the liver image study and the identifier for the chest image study. Due to the selections, the clinical items display region 406 includes a liver image 410 from the liver image study and a chest image 412 from the chest image study.

Referring now to FIG. 4E, the GUI 236 is depicted after the client universal clinical viewer 104 receives selections of the identifier for the liver image study and the identifier for stomach cancer clinical report. Due to the selections, the clinical items display region 406 includes a liver image 410 from the liver image study and the stomach cancer clinical report.

Although the above-described technologies have been described above in the context of healthcare, other possibilities are contemplated. According to embodiments, the templates include tags and rules that are related to areas other than healthcare, such as education, aspects of an enterprise, etc. According to the embodiments, non-clinical data items are matched to the templates based upon the rules of the templates. According to the embodiments, the nodes and edges of the knowledge graph represent relationships between the non-healthcare related templates. According to the embodiments, the above-described processes are utilized to cause graphical data corresponding to tagged non-clinical data items to be presented on a display.

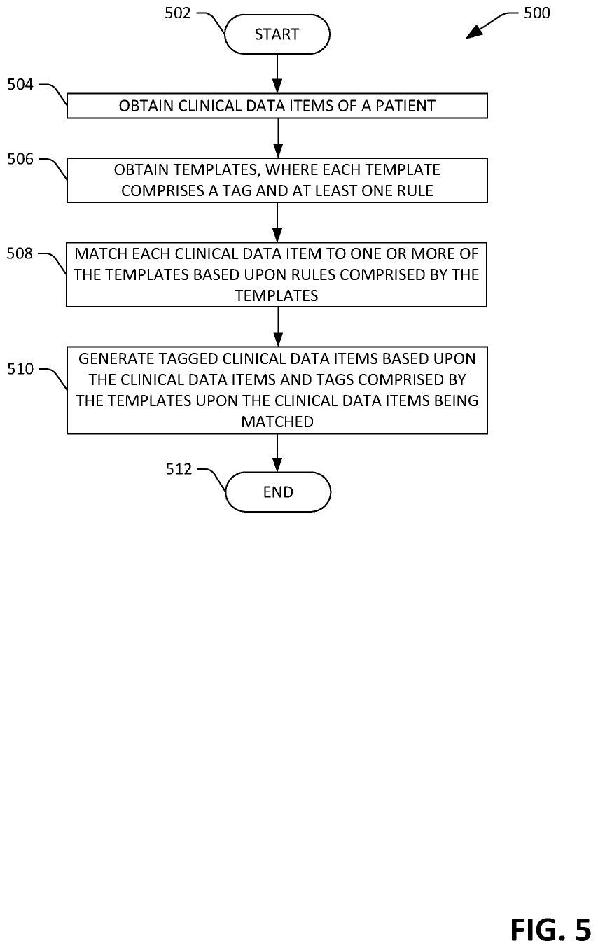

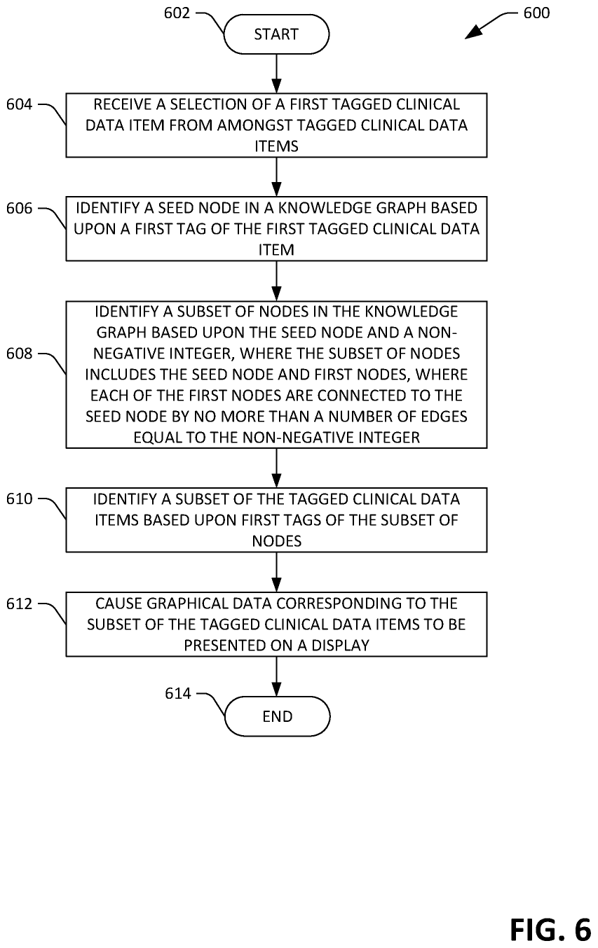

FIGS. 5 and 6 illustrate exemplary methodologies relating to classification of clinical data items based upon knowledge graphs. While the methodologies are shown and described as being a series of acts that are performed in a sequence, it is to be understood and appreciated that the methodologies are not limited by the order of the sequence. For example, some acts can occur in a different order than what is described herein. In addition, an act can occur concurrently with another act. Further, in some instances, not all acts may be required to implement a methodology described herein.

Moreover, the acts described herein may be computer-executable instructions that can be implemented by one or more processors and/or stored on a computer-readable medium or media. The computer-executable instructions can include a routine, a sub-routine, programs, a thread of execution, and/or the like. Still further, results of acts of the methodologies can be stored in a computer-readable medium, displayed on a display device, and/or the like.

Referring now to FIG. 5, a methodology 500 performed by a computing system for tagging clinical data items is illustrated. The methodology 500 begins at 502, and at 504, the computing system obtains clinical data items of a patient. At 506, the computing system obtains templates. Each template comprise a tag and at least one rule. At 508, the computing system matches each clinical data item to one or more of the templates based upon rules comprised by the templates. At 510, the computing system generates tagged clinical data items based upon the clinical data items and tags comprised by the templates upon the clinical data items being matched. The tagged clinical data items comprise tags corresponding to the tags comprised by the templates. The methodology 500 concludes at 512.

Turning now to FIG. 6, a methodology 600 performed by a computing system that facilitates displaying relevant clinical data items is illustrated. The methodology 600 begins at 602, and at 604, the computing system receives a selection of a first tagged clinical data item from amongst tagged clinical data items. The tagged clinical data items are generated based upon clinical data items from a plurality of electronic sources being matched to templates stored in a data store. Each template comprises a tag and at least one rule. Rules of the templates are matched to the clinical data items, thereby generating the tagged clinical data items. At 606, the computing system identifies a seed node in a knowledge graph stored in the data store based upon a first tag of the first tagged clinical data item. The knowledge graph comprises nodes and edges connecting the nodes. The nodes represent the plurality of templates and the edges represent relationships between the templates. Each node comprises a respective tag corresponding to a respective template in the templates. At 608, the computing system identifies a subset of nodes in the knowledge graph based upon the seed node and a non-negative integer. The subset of nodes includes the seed node and first nodes. Each of the first nodes are connected to the seed node by no more than a number of edges equal to the non-negative integer. At 610, the computing system identifies a subset of the tagged clinical data items of the patient based upon first tags of the subset of nodes. At 612, the computing system causes graphical data corresponding to the subset of the tagged clinical data items to be presented on a display. The methodology 600 concludes at 614.

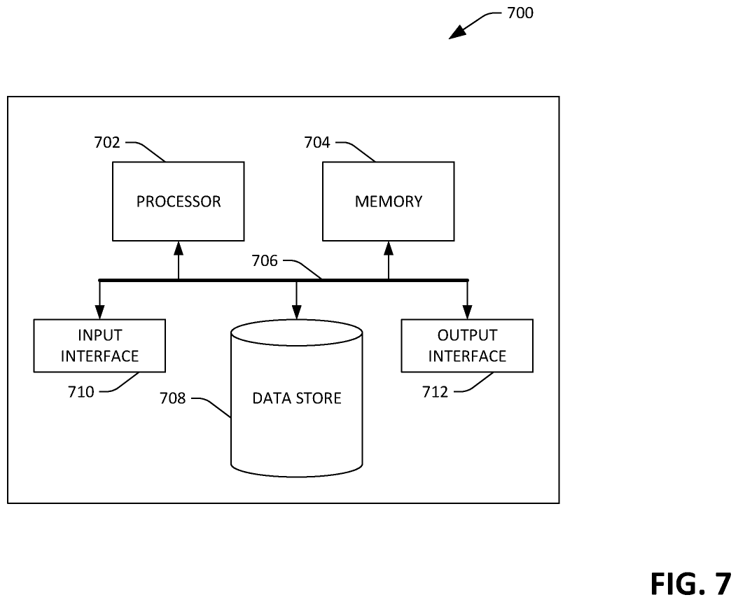

Referring now to FIG. 7, a high-level illustration of an exemplary computing device 700 that can be used in accordance with the systems and methodologies disclosed herein is illustrated. For instance, the computing device 700 may be used in a system that tags clinical data items of a patient. By way of another example, the computing device 700 can be used in a system that displays tagged clinical data items of a patient. The computing device 700 includes at least one processor 702 that executes instructions that are stored in a memory 704. The instructions may be, for instance, instructions for implementing functionality described as being carried out by one or more components discussed above or instructions for implementing one or more of the methods described above. The processor 702 may access the memory 704 by way of a system bus 706. In addition to storing executable instructions, the memory 704 may also store clinical data items, tagged clinical data items, templates (including tags and rules), knowledge graphs, adapters, etc.

The computing device 700 additionally includes a data store 708 that is accessible by the processor 702 by way of the system bus 706. The data store 708 may include executable instructions, clinical data items, tagged clinical data items, templates (including tags and rules), knowledge graphs, adapters, etc. The computing device 700 also includes an input interface 710 that allows external devices to communicate with the computing device 700. For instance, the input interface 710 may be used to receive instructions from an external computer device, from a user, etc. The computing device 700 also includes an output interface 712 that interfaces the computing device 700 with one or more external devices. For example, the computing device 700 may display text, images, etc. by way of the output interface 712.

It is contemplated that the external devices that communicate with the computing device 700 via the input interface 710 and the output interface 712 can be included in an environment that provides substantially any type of user interface with which a user can interact. Examples of user interface types include graphical user interfaces, natural user interfaces, and so forth. For instance, a graphical user interface may accept input from a user employing input device(s) such as a keyboard, mouse, remote control, or the like and provide output on an output device such as a display. Further, a natural user interface may enable a user to interact with the computing device 700 in a manner free from constraints imposed by input devices such as keyboards, mice, remote controls, and the like. Rather, a natural user interface can rely on speech recognition, touch and stylus recognition, gesture recognition both on screen and adjacent to the screen, air gestures, head and eye tracking, voice and speech, vision, touch, gestures, machine intelligence, and so forth.

Additionally, while illustrated as a single system, it is to be understood that the computing device 700 may be a distributed system. Thus, for instance, several devices may be in communication by way of a network connection and may collectively perform tasks described as being performed by the computing device 700.

Various functions described herein can be implemented in hardware, software, or any combination thereof. If implemented in software, the functions can be stored on or transmitted over as one or more instructions or code on a computer-readable medium. Computer-readable media includes computer-readable storage media. A computer-readable storage media can be any available storage media that can be accessed by a computer. Such computer-readable storage media can include random-access memory (RAM), read-only memory (ROM), electrically erasable programmable read-only memory (EEPROM), compact disc read-only memory (CD-ROM) or other optical disk storage, magnetic disk storage or other magnetic storage devices, or any other medium that can be used to store desired program code in the form of instructions or data structures and that can be accessed by a computer. Disk and disc, as used herein, include compact disc (CD), laser disc, optical disc, digital versatile disc (DVD), floppy disk, and blu-ray disc (BD), where disks usually reproduce data magnetically and discs usually reproduce data optically with lasers. Further, a propagated signal is not included within the scope of computer-readable storage media. Computer-readable media also includes communication media including any medium that facilitates transfer of a computer program from one place to another. A connection can be a communication medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio and microwave are included in the definition of communication medium. Combinations of the above should also be included within the scope of computer-readable media.

Alternatively, or in addition, the functionally described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim.

What is claimed is:

1. A computing system, comprising:
a processor;
a data store comprising:
templates, wherein each template in the templates comprises a tag and at least one rule; and
a knowledge graph, wherein the knowledge graph comprises nodes and edges connecting the nodes, wherein the nodes represent the templates and the edges represent relationships between the templates, wherein each node comprises a respective tag corresponding to a respective template in the templates; and
memory storing instructions that, when executed by the processor, cause the processor to perform acts comprising:
obtaining clinical data items of a patient;
matching each clinical data item in the clinical data items to one or more templates in the templates based upon the at least one rule of the templates;
generating tagged clinical data items based upon the clinical data items and tags of the templates for the clinical data items being matched;
upon receiving a keyword, identifying a seed node in the knowledge graph based upon the keyword;
identifying a subset of nodes in the knowledge graph based upon the seed node and a non-negative integer, wherein the subset of nodes includes the seed node and first nodes, wherein each of the first nodes are connected to the seed node by no more than a number of edges equal to the non-negative integer;
identifying a subset of the tagged clinical data items based upon first tags of the subset of nodes; and
causing graphical data corresponding to the subset of the tagged clinical data items to be presented on a display.

2. The computing system of claim 1, wherein the clinical data items are obtained over a network connection from a plurality of electronic sources.

3. The computing system of claim 1, wherein the clinical data items include Digital Imaging and Communications in Medicine (DICOM) clinical data items and non-DICOM clinical data items.

4. The computing system of claim 3, the acts further comprising:
prior to matching each clinical data item in the clinical data items to the one or more templates in the templates, converting the non-DICOM clinical data items to DICOM attributes by way of an adapter.

5. The computing system of claim 1, wherein the clinical data items include one or more of:
a historical imaging study;
a clinical report;
an admission form;
a radiation report;
a result obtained as output of an algorithm or artificial intelligence (AI) model;
a measurement;
a video; or
a clinical portable document format (PDF) document.

6. The computing system of claim 1, wherein the clinical data items comprise first Digital Imaging and Communications in Medicine (DICOM) attributes, wherein the first DICOM attributes of the clinical data items are matched to second DICOM attributes specified by the rules of the templates.

7. The computing system of claim 1, wherein the graphical data comprises identifiers for each of the subset of the tagged clinical data items.

8. The computing system of claim 1, wherein the subset of the tagged clinical data items are images of the patient, wherein the graphical data includes the images.

9. The computing system of claim 1, wherein the subset of nodes include the seed node, a first node, and a second node, wherein the seed node and the first node are connected by a first edge, wherein the first node and the second node are connected by a second edge, wherein the first node represents a body part template corresponding to a body part, wherein the second node represents a disease template corresponding to a disease that affects the body part.

10. The computing system of claim 1, wherein the edges of the knowledge graph are directed edges.

11. The computing system of claim 1, wherein the acts occur responsive to receiving an identifier for the patient from a computing device operated by a healthcare worker.

12. The computing system of claim 1, wherein each of the subset of the tagged clinical data items includes an associated date, wherein the graphical data comprises a timeline that includes identifiers for each of the subset of the tagged clinical data items, wherein the timeline is chronologically arranged based upon the associated date of each of the subset of the tagged clinical data items.

13. The computing system of claim 1, the acts further comprising:
receiving a medical record number (MRN) of the patient, wherein the clinical data items of the patient are obtained based upon the MRN of the patient.

14. A method executed by a processor of a computing system, the method comprising:
receiving a selection of a first tagged clinical data item of a patient from amongst tagged clinical data items of the patient, wherein the tagged clinical data items are generated based upon clinical data items from a plurality of electronic sources being matched to templates stored in a data store, wherein each template comprises a tag and at least one rule, wherein rules of the templates are matched to the clinical data items, thereby generating the tagged clinical data items;
identifying a seed node in a knowledge graph stored in the data store based upon a first tag of the first tagged clinical data item, wherein the knowledge graph comprises nodes and edges connecting the nodes, wherein the nodes represent the templates and the edges represent relationships between the templates, wherein each node comprises a respective tag corresponding to a respective template in the templates;
identifying a subset of nodes in the knowledge graph based upon the seed node and a non-negative integer, wherein the subset of nodes includes the seed node and first nodes, wherein each of the first nodes are connected to the seed node by no more than a number of edges equal to the non-negative integer;
identifying a subset of the tagged clinical data items of the patient based upon first tags of the subset of nodes; and
causing graphical data corresponding to the subset of the tagged clinical data items to be presented on a display.

15. The method of claim 14, wherein a rule of a first template comprises evaluation criteria and a comparison value, wherein the first tagged clinical data item is generated by evaluating a first clinical data item against the evaluation criteria and the comparison value.

16. The method of claim 14, wherein the subset of the tagged clinical data items includes an image of the patient and a clinical report about the patient, wherein the graphical data includes an identifier for the image and an identifier for the clinical report.

17. The method of claim 14, wherein each template in the templates represents:
a body part;
a medical facility;
a medical department within the medical facility;
an imaging modality;
a piece of medical equipment; or
a disease.

18. A non-transitory computer-readable storage medium comprising instructions that, when executed by a processor of a computing system, cause the processor to perform acts comprising:
obtaining clinical data items of a patient;
matching each clinical data item in the clinical data items to one or more templates in templates stored in a data store based upon rules included in the templates;
generating tagged clinical data items based upon the clinical data items and tags included in the templates upon the clinical data items being matched;
receiving a keyword from a computing device operated by a healthcare worker;
identifying at least one seed node in a knowledge graph stored in the data store based upon the keyword, wherein the knowledge graph comprises nodes and edges connecting the nodes, where the nodes represent the templates and the edges represent relationships between the templates, wherein each node comprises a respective tag corresponding to a respective template in the templates;
identifying a first subset of the tagged clinical data items based upon a tag of the at least one seed node, wherein each of the first subset of the tagged clinical data items includes the tag of the seed node;
causing first graphical data corresponding to the first subset of the tagged clinical data items to be presented on a display of the computing device;
upon receiving an indication from the computing device, identifying a subset of nodes in the knowledge graph, wherein each node in the subset of nodes is connected to the at least one seed node via an edge;
identifying a second subset of the tagged clinical data items based upon the subset of nodes; and
causing second graphical data corresponding to the second subset of the tagged clinical data items to be presented on the display concurrently with the first graphical data.

19. The non-transitory computer-readable storage medium of claim 18, the acts further comprising:
prior to obtaining the clinical data items, receiving a user-defined template as input from a user, wherein the user-defined template comprises a user-defined tag and at least one user-defined rule;
receiving a selection of a node in the nodes of the knowledge graph; and
generating a user-defined node in the knowledge graph based upon the user-defined template, wherein the user-defined node is connected to the node in the knowledge graph.

20. The non-transitory computer-readable storage medium of claim 18, the acts further comprising:
subsequent to generating the tagged clinical data items and prior to receiving the keyword, causing identifiers for the tagged clinical data items to be presented on the display, wherein an identifier for a first tagged clinical data item is selected by the healthcare worker, wherein the identifier for the first tagged clinical data item is the keyword.
